(19) United States
(12) Patent Application Publication
(10) Pub. No.: US 2025/0135178 A1
(43) Pub. Date: May 1, 2025

(54) EXTENSION SYSTEM FOR COUPLING TUBULAR DEVICES

(71) Applicant: Petrus Antonius Besselink, Enschede (NL)

(72) Inventor: Petrus Antonius Besselink, Enschede (NL)

(21) Appl. No.: 18/715,185

(22) PCT Filed: Dec. 1, 2022
(86) PCT No.: PCT/IB2022/000738

§ 371 Date: Nov. 11, 2024

Related U.S. Application Data
(60) Provisional application No. 63/284,736, filed on Dec. 1, 2021.

(51) Int. Cl. A61M 39/10; A61M 25/09

(52) U.S. Cl. CPC A61M 39/1011; A61M 25/0905

(57) ABSTRACT

An extension system for connecting or coupling a guidewire, primary or initial wire to an extension or secondary wire, a method of coupling elongate devices (40,41,42,43) and a method of using an extension system. The extension system is formed by the connection of devices to extension tubes or wires. In this way, connecting or coupling a guidewire, primary or initial wire to an extension or secondary wire may be done during or before a medical procedure. The resulting coupling from the extension system allows independent relative movement between inner and outer components of an endoluminal device such as a guiding catheter, endoscope, medical instrument or guidewire. In an assembled state, multiple concentric wires and tubular components can be locked and secured by a simple single movement, followed by a relative rotation, translation or both in order to create a mechanical strength between them that resembles such strength as if there was no connection at all. The extension system may also be used for procedures other than medical procedures.

### TECHNICAL FIELD

The present specification generally relates to the field of connecting solid or tubular devices to extension tubes or wires, and more particularly to the connection of such devices to extension tubes or wires to form an extension system for connecting or coupling a guidewire, primary or initial wire to an extension or secondary wire during or before a medical procedure.

### BACKGROUND ART

There are numerous circumstances where a thin, elongate device must be inserted into a lengthy, narrow and often curved or branched tubular member in order to effect navigation and related repair, insertion or other complex activities associated with the device. With particular regard to minimally-invasive medical procedures, there is a need for flexible and steerable guidewires (also referred to herein as guidewire assemblies), stylets, catheters and related devices that generally have to be maneuvered through tortuous body lumens through one or more of pushing, pulling and tangential rotation, and more particularly do so by transferring such movements initiated at the proximal end of the device as accurately as possible to the distal end.

In one example, guidewires or guiding catheters are used for the purpose of positioning catheters in exploratory procedures, diagnosis and treatment of various medical conditions. While conventional guidewire assemblies with a steerable tip are known in the art as a way to achieve some degree of maneuverability, all have some form of drawback. For example, attempting to lengthen guidewires through the connecting of different materials by welding or gluing may be impossible or create other problems during use.

Once in place, the guidewire provides the means to place a non-steerable device, such as a catheter, at the chosen body site. For example, a catheter is slid over the guidewire until the catheter, or some working portion thereof, is positioned within the vasculature at the desired location. Generally speaking, guidewires of a standard length are longer than the non-steerable devices with which they are used to permit independent movement of the device and the wire.

Blackledge discloses in U.S. Pat. No. 6,491,646 that guidewires are used in various medical procedures to position medical devices at desired locations within a patient's vascular system. Steerable guidewires are inserted and maneuvered through the patient's vasculature to a previously chosen location.

Angioplasty is one surgical application where a guidewire is often used. In angioplasty a dilatation catheter having an inflatable balloon structure is used to compress occlusive or blockage material against the sides of a vessel, thereby permitting (ideally) circulation to be re-established. In preparatory procedures, the site of a vascular restriction, occlusion or stenosis is identified. The guidewire is inserted into the patient's femoral artery and maneuvered or steered to the location of the restriction. Insertion of the guidewire is facilitated by a video X-ray device which allows the surgeon to observe the movement of the guidewire's distal tip. The guidewire distal tip generally comprises a radiopaque metal to enhance X-ray viewing. A dilatation catheter then is inserted over the guidewire so that its working segment is located adjacent the restriction. Generally this means that the catheter balloon is positioned adjacent the vascular restriction or blockage.

During a simple angioplasty procedure, the dilatation catheter balloon is inflated to open the restriction, and then is removed along with the guidewire. However, complications sometimes arise which prevent the surgeon from completing this simple procedure.

Occasionally the balloon catheter malfunctions. Sometimes a larger (or smaller) balloon is required further to dilate the vascular restriction, or another device or other type of catheter is needed to remove vascular material.

In the usual procedure to exchange catheters, the guidewire is removed from the patient leaving the catheter in the vascular system. An exchange wire is inserted through the catheter and the catheter removed, leaving the exchange wire in place. The new catheter is inserted over the exchange wire and the exchange wire removed and replaced with the guidewire.

It is desirable to keep the guidewire in the patient's vasculature for various reasons. However, the primary reason for not removing the guidewire is that the initial placement of the guidewire requires extensive, time consuming manipulation. Removal and repositioning of the guidewire would be equally time consuming, possibly requiring further exposure to drugs, exposure to additional radiation, and, in general, infliction of additional trauma to the patient.

In those cases where catheter exchange is desired, the surgeon could simply remove the catheter over the guidewire, leaving the guidewire in the patient. However, to facilitate the removal and replacement of a catheter, the guidewire must be sufficiently long to allow the surgeon to grip a portion of the wire as the catheter is being withdrawn. This requires the guidewire to be long enough to provide an external portion longer than the catheter in addition to the portion remaining in the patient.

Unfortunately, a guidewire of sufficient length has inferior handling characteristics, thereby making more difficult the steering and maneuvering manipulations needed for guidewire placement. The added length also imposes itself on the usually cramped surgical arena thereby causing distractions to other surgical support activities.

It is for these reasons that guidewires are usually only slightly longer than balloon catheters, e.g. 20-50 centimeters longer, and that a much longer exchange wire (and exchange procedure) is used. In other words, the guidewire-extension wire combination has a length approximating that of an exchange wire. Illustrating the above, a dilatation catheter has a length in the range of about 130 cm to about 160 cm, a suitable guidewire would have a length in the range of about 160 cm to about 200 cm and an exchange wire would have a length in the range of about 260 cm to about 340 cm. As will be understood, utilization of an exchange wire in an exchange wire procedure is complicated and time-consuming.

A recent development involves coupling or connecting a second length of wire, sometimes called an extension wire or secondary wire, to the exposed, proximal end of a guidewire. The second wire length should be of sufficient length to allow the catheter to be withdrawn while leaving the guidewire in the patient. Various approaches have been suggested for effecting the attachment of an extension wire to a guidewire.

In one approach, such as described in U.S. Pat. No. 4,922,923 to Gambale et al., a guidewire and an extension are joined together by crimping using a dedicated tool. A drawback of this approach is that once the wires have been crimped, the connection therebetween is substantially permanent, and the extension wire cannot be detached from the guidewire except by severing it, such as by cutting.

Instead of crimping the guidewire to the extension wire, attempts have been made to engage the extension wire to the guidewire frictionally. Such attempts are described, for example, in U.S. Pat. No. 5,113,872 to Jahrmarkt et al. and related U.S. Pat. No. 5,117,838 to Palmer et al. both of which disclose a guidewire extension system in which the distal end of the extension wire comprises a small diameter tube in which there is disposed a small diameter, open pitch, flat wire coiled spring. The proximal end of the guidewire has a reduced diameter portion which is inserted into the tube assembly to complete the connection. The reduced diameter proximal end of the guidewire is slightly larger than the internal diameter of the coiled spring of the extension wire, thereby creating a frictional engagement when one is inserted into the other. A swivel joint for minimizing twisting of the extension guidewire when connecting or disconnecting it from the extension wire would be very difficult to manufacture reliably and furthermore appears to require an alignment tool to ease insertion.

U.S. Pat. No. 4,875,489 to Messner et al. discloses an extendable guidewire in which concentric tubular segments are secured to one or the other of the sections to be connected. The inner tubular segment has a longitudinal slot therein which permits it to expand when a cooperating male portion is inserted therein. The outer tubular member of the connector assembly restricts the expansion of the inner tubular member as the male portion is inserted therein.

U.S. Pat. No. 4,846,193 to Tremulis et al. discloses a guidewire having first and second telescopically extendable sections movable between axially extended and retracted positions. No disengagement of the guidewire and extension wire is disclosed.

U.S. Pat. No. 4,966,136 to Kraus et al. discloses an internally threaded female connection member secured to the distal end of the extension wire. The internally threaded female connection member is disclosed to be freely rotatable with respect to the extension wire by securement thereto by means of a collar. The body of the extension wire has a distal enlargement which cooperates with the collar to permit it to be freely rotated. The female connection member of the extension wire cooperates with a threaded male portion located on the proximal end of the guidewire. Unfortunately, this mechanism sacrifices pushability and flexibility as the ball and socket joint do not effect a mechanical lock sufficiently to transmit such desirable properties.

U.S. Pat. No. 4,827,941 to Taylor et al. discloses a guidewire extension system employing a tubular female connector portion on one wire and a cooperating male portion on the other. The connecting male portion has an effective diameter in one radial dimension which is slightly larger than the inner diameter of the tubular portion, where the guidewire uses an undulating shape in order to create an interference fit.

The aforementioned guidewire extension systems all have drawbacks, particularly as they relate to excess ease of engagement or disengagement. Moreover, in situations where frictional engagement is used to overcome the disadvantages of permanent approaches such as crimping, disengagement may nonetheless occur. Furthermore, problems of kinking at the connection have been experienced with some systems.

In addition, prior extendable wires for use in coronary angioplasty procedures have been found to be unsuitable in peripheral arteries because the connections are not strong enough. Further, some connections have larger diameters than the rest of the guidewire system. This may cause snagging of the catheter. It also means that the catheter with which such connection system is used must have a larger internal diameter than would be necessary were a smaller diameter coupler employed.

In U.S. Pat. Nos. 10,441,746, 11,241,557, PCT/US22/49567 and U.S. Provisional Applications 63/278,743, 63/337,709 and 63/397,050 the author of the present disclosure describes guidewires or catheters that are built from several concentric components that make a steerable device by moving these components relatively in axial and tangential direction. One such device is a described as a Double Torque Motor (DTM). For the DTM, it is important that outer tube, inner tube and pull wire can slide freely over and through each other with a friction that is as low as possible. Any deviation from the cylindrical surfaces will locally cause difficulties, while the proximal actuation of the distal steering in revolving and bending may result in sub-optimal functioning. Moreover, extension of such devices is cumbersome with conventional technology.

Accordingly, a need exists for an extension system for connecting or coupling a guidewire, primary or initial wire to an extension or secondary wire such that the extension system allows independent relative movement of inner components relative to outer components. A need also exists for a way to simplify catheter exchange and eliminate the need to use an exchange wire. An additional need exists to extend a guidewire when a catheter or related device has to be removed and replaced with another. Yet another need exists to provide a guidewire extension system which minimizes the possibility of system failure by kinking or undesirable bending at the connection between the guidewire and the extension wire. A need further exists to provide a guidewire extension system which does not require the guidewire or extension wire to be rotated when attaching one to the other. Yet another need exists for a method to connect the different components in such a way that the outer and inner surfaces of the tubular components remain smooth, including at their place of coupling. A further need exists for such an extension system that is useful for various procedures in general, including those unrelated to the aforementioned medical procedures some of which that will be described in more detail as follows.

### DISCLOSURE OF INVENTION

In general, the extension system of the present disclosure is a set (or assembly) of concentric components with locks, which can be used for coupling elongate devices (whether as solid wires or tubular members) to extensions that are either solid or tubular for all kinds of applications in the medical field, as well as in the non-medical field. This type of locks enables the coupling of multiple concentric devices in a single procedure. These and additional features provided by the embodiments described herein will be more fully understood in view of the following detailed description, in conjunction with the drawings.

The author of the present disclosure notes with particularity that the various embodiments disclosed herein may be used in combination with the device of the present disclosure or with components thereof. Moreover, the contents disclosed in U.S. Pat. Nos. 10,441,746, 11,241,557, PCT/US22/49567 and U.S. Provisional Applications 63/278,743, 63/337,709 and 63/397,050 are hereby incorporated by reference in their entirety into the present disclosure.

In one aspect, the extension system is used for connecting the proximal end of an endoluminal device (such as a guiding catheter, endoscope, medical instrument, guidewire or other elongate element) to the distal end of an extension wire or tube. In accordance with the present disclosure, the system comprises engagement means, such as shape-fit locks located on the proximal end, and distal end, respectively, of a guidewire and an extension wire or tube. The connection is secured by a second set of tubes that surrounds the first set and also has shape-fit locks, but placed on one or both of a different axial or tangential location. Assembly and disassembly can be achieved by placing all locks simultaneously in the same axial location and rotating all locks into the right position as well. As soon as the connection is secured, the assembly can take axial forces, torque and bending, while the inner and outer sets can still be relatively moved tangentially and axially.

In another aspect, a guidewire extension system for connecting or coupling a guidewire, primary or initial wire to an extension or secondary wire during or before a medical procedure is disclosed. The resulting coupling from such a system allows independent relative movement from the inner components relative from the position of the outer components.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 shows an elongate solid wire with a lock at its end;

FIG. 2 shows a tube with a lock at its end;

FIG. 3 shows a schematic drawing of two tubes of FIG. 2 assembled, while the respective locks are engaged;

FIG. 4 shows two sets of separated tubes with different diameters, sized to be assembled with the first set with small diameter sliding inside the second set with larger diameter, where both sets have the locks depicted in FIG. 3;

FIG. 5 shows both sets of FIG. 4 just prior to assembly;

FIG. 6 shows the first and second tube in their assembled stable state, with all locks in the same axial location;

FIG. 7 shows a schematic view on the entire device of FIG. 6, wherein a part of the outer tube on the right side is partially opened to show the position of the lock that is formed by the inner tubes;

FIG. 8 shows a schematic drawing of two tubes, similar to the tubes in FIG. 3, assembled, while the respective locks are engaged, wherein the lock edges have a curved contour for improving the strength of the connection;

FIG. 9 shows two sets of separated tubes with different diameters, each with the curved contour of FIG. 8 just prior to assembly into locked tube sets; and

FIG. 10 shows a schematic view on the entire device of FIG. 9 with curved lock contour after assembly, wherein a part of the outer tube on the right side is partially opened to show the position of the lock that is formed by the inner tubes.

### MODES FOR CARRYING OUT THE INVENTION

Various features and advantageous details are explained more fully with reference to the non-limiting embodiments that are illustrated in the accompanying drawings and detailed in the following description.

An object of the present disclosure is that multiple concentric tubular components can be locked and secured by a simple single movement, followed by at least one of a relative rotation and relative translation, which creates a continuous, rigid mechanical strength as if there was no connection at all.

It is still a further object of the present disclosure to provide a guidewire extension system which has substantially the same flexibility on bending and pushability at its connection site, as compared to the remainder of the length of the guidewire.

It is yet another object of the present disclosure to provide a unitized guidewire extension system having a uniform, smooth, continuous outer diameter along the guidewire, connector, and extension wire. Methods of manufacturing an extension system of this disclosure and methods of using a system of this disclosure also are disclosed.

It is another object of the present disclosure that tubes or wires of different materials can be connected in a simple and reliable way.

In another object of the disclosure two sets of concentric tubes can be connected and secured by moving the locks in the inner tube away from the locks in the outer tube axially or by rotation tangentially over an angle that differs from a multiple of 360 degrees.

It is another object of the disclosure that more than two sets of concentric wires and tubes are locked.

In another object of the disclosure the inner lumen of the assembled device has a smooth, cylindrical surface that allows the easy insertion of additional devices from the proximal side to the distal side.

It is another object of the disclosure that the assembled device has a smooth, cylindrical outer surface that allows the easy insertion of additional devices from the proximal side to the distal side over its outer surface.

It is still another object of the present disclosure that the connection of proximal and distal sides of the assembly is stable enough to transmit pulling and pushing forces as well as bending and torque over the length of the entire device, while the possibility of axial sliding and tangential rotation between inner wire or tube and outer tube remains possible.

Another object of the present disclosure is that the connection by the locks may be made permanent by placing at least one spot weld, glue joint, crimp or any other connection between inner and outer component on any remote location, away from the location of the lock joints. If it is needed to disconnect the locks, a remote releasable clamping device may be used instead of a weld, crimp or glue joint.

In another object of the disclosure markers are used on the surface of inner and outer component to show the relative position that enables assembly or disassembly of the joints.

It is another object of the disclosure that the length of distinctive layers of tubes does not have to be equal. For example, an outer set of tubing may reach until close to a common proximal end of the assembly, while a distal end of an inner set of tubing may protrude over a considerable length as compared to the outer set. This may be the case for a guidewire with a distal tip that bends by actuation by means of an additional internal pull wire that runs from tip to the proximal end. On the proximal side there may be a need for a tool that can grip inner tube, outer tube and pull wire separately.

This application relates to the field of producing and using an improved and simple device coupling elongate devices (whether solid or tubular) to extensions (also whether solid or tubular).

In one form, the guidewire extension system utilizes a turnbuckle configuration which does not require that either the guidewire or extension wire be rotated when attaching one to the other. Such a configuration is beneficial in that it allows the guidewire be held stationary while in certain movement-sensitive parts of the vasculature (such as a patient's blood vessel) where unnecessary movement can induce trauma. It is also beneficial to have the majority of the length of the extension wire held stationary during the connection process, as having the extension wire be self-contained in its tubular carrier package allows medical personnel to concentrate upon engaging the two wires, thereby avoiding the awkwardness of having an uncontained extension wire. Furthermore, the construction of the extension system described herein allows a proximal end thereof to be located outside the body of the patient while the distal end is located inside the body of the patient.

The advantages of the various embodiments of the device disclosed herein will become more apparent after reference to the following description, wherein some embodiments are elucidated. For the purpose of this description the words locking, connecting and mating are supposed to be resulting in similar functions, but may be used through each other for clarity. Mating may be used for fitting two opposing locks in order to connect two separate devices or components, but it is not strictly distinguished here. Other terms that refer to similar effects are clicking, pressing or inserting, to name a few, and all these effects are supposed to be included in this application. The lock itself may be described as a hinge, because it allows some flexibility if it is not secured by forming a stabilized connection, such as through joining each of the first and second sets of tubes, forming them into an assembly that makes up an extension system and then moving the first and second sets relative to one another.

Techniques for manufacturing the locks include machining, grinding, cutting, etching, wire erosion, electrical discharge machining (EDM), photo-etching or any other means of making the desired shape, and are referred to here as “cutting”.

There are several options and combinations to use the embodiments as disclosed hereafter.

In one form, a jacket, sleeve or related covering may be formed over the coupling area of the outer members after assembly in order to enhance fluid or gas tightness of the device. Such a jacket can be made of a sleeve, tubing, glue, a UV-curing polymer or a heat shrinkable polymer, to name a few.

In the previously-mentioned U.S. Pat. Nos. 10,441,746, 11,241,557, as well as PCT application PCT/US22/49567 and U.S. Provisional Applications 63/278,743, 63/337,709 and 63/397,050, a catheter or guidewire is disclosed with a helical cut in the tubular wall. In response of changing the length of this helical section by a central pull wire or tube, the result is a controlled tangential revolving around its own length axis of the distal end without the need of having proximal rotation. Optimization of the used material, dimensions of the tube wall, slot width of the helix, the pitch and the number of helical loops leads to a tube that revolves over several turns, and upon unloading it turns back to its original state with sufficient torque output to use for cutting/reaming purposes. When a second tube surrounds the first one and has also a similar helical cut, but now in clockwise direction, it would revolve in a direction opposite from the revolving of the first tube.

But if both tubes are connected at their distal ends, while the proximal ends can be moved axially in relation to each other, the tendency to revolve will be in the same direction. This is caused by the fact that tension in length of one tube will automatically cause compression of the length of the other tube. Now the output rotation is more pronounced and the output torque—that is to say, the aforementioned DTM—is larger as well. Such a set of cooperating tubes may be made from a special material like binary or ternary superelastic or linear elastic Nitinol alloys, but other materials and combinations of materials may be used as well.

In the manufacturing of such a device it is favorable to have it as short as is reasonably possible. This is better for pricing of the base material, as well as for handling the components during laser cutting, for example. Handling during use in the catheter laboratory (i.e., cathlab) is easier for a short guidewire than for a longer one. However, during use in a cathlab it may be necessary to have a considerable extra length for having the possibility to slide additional devices over the guidewire, like a guiding catheter or a balloon catheter (eventually with a stent loaded on the balloon). Such an extra length can be generated, simply by making the entire device long enough out of components of one single material, although such excess length may result in the aforementioned disadvantages.

Another option is the use of different components (including different materials), which are assembled into a complete device. One example is a Nitinol distal section, with improved flexibility and steerability, mounted on a relatively long and more rigid proximal section that could be made out of a different material, such as out of stainless steel. Such an approach can help to keep both raw material and manufacturing costs low. In one form, both sections may be connected by crimping, glue, welding, soldering to name a few, taking into consideration additional considerations when Nitinol is being welded to steel.

Another problem is that manufacturing of a device with several axially independently movable concentric components, like an outer tube, inner tube and pull wire is almost impossible when the abovementioned techniques are used for a guidewire with small diameter.

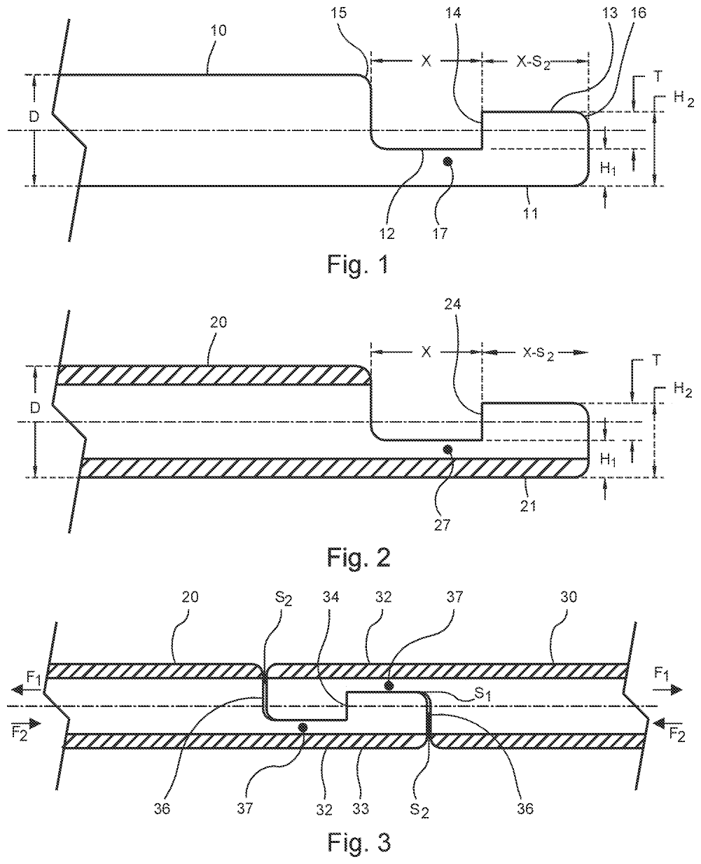

Referring first to FIG. 1, one end of a first wire 10 is shown with diameter D and a coupling section 11 is formed at one end. As will be understood, by being configured as a wire, it has a solid (rather than tubular) cross-sectional profile and related structure. The first wire 10 forms an elongate member that may be used in the disclosed extension system when connected to a second (extension) wire that is not presently shown. In one form, the coupling section 11 is created by cutting a part of the material out. One portion 12 of the coupling section 11 forms a deep cut and leaves a remaining layer 17 of first wire 10 with a height H1, which is smaller than D/2. Another portion 13 is cut to a height H2 which is more than D/2. An edge 14 with a height T and that is perpendicular to the length axis is present between portions 12 and 13. The height T of edge 14 equals or is slightly less than H1 minus H2, depending on the manufacturing precision and the desirable amount of slack that is needed for proper locking with such second wire (or an extension tube, as will be discussed in more detail as follows). In one form, edges 15 and 16 may be rounded slightly for ease of insertion, but edge 14 should be as flat as possible for optimal mechanical strength.

The axial length X of portion 12 should be slightly more than length X-S2 (where S2 is an axial slack) for portion 13, in order to enable the coupling with the second wire or tube, wherein portion of such second wire (which is similar to portion 13 of the first wire 10) fits into section 12 of the first wire 10, while portion 13 of the first wire 10 fits into a section of the second wire that is similar to section 12. Therefore, the axial slack S2 should be just large enough to achieve an easy, but reliable locking.

Referring next to FIG. 2, a tube (also referred to herein as a tubular member or tubular elongate member) 20 with diameter D is shown with a coupling section 21 that is cut or otherwise formed therein. As can be seen, coupling section 21 is generally similar to the coupling section 11 of FIG. 1. The tube 20 may be connected to the wire 10 of FIG. 1, or to a similar tube that will be shown in FIG. 3. It may even be connected to extension wires or extension tubes with a diameter that differs from diameter D of tube 20, as long as an edge 24 makes enough contact with the opposing edge of the extension wire or tube.

Referring next to FIG. 3, the tube 20 and a companion tube 30 (which in one form may qualify as the second (extension) tube that was briefly mentioned previously) are shown being assembled to be placed in registration with one another such that a common lock 33 is formed. As can be seen, the lock 33 is made out of respective coupling sections 32 in a manner similar to the coupling section 21 of FIG. 2. Within the present disclosure, the registration includes having compatible surfaces (such as through geometry, sizing or the like) of the tubes 20 and 30 be properly aligned with a slight axial offset to have their respective coupling sections be radially facing one another. In this way, when the facingly-adjacent coupling sections are brought into contact with one another along their mutual radial direction, the elongate members form an interference fit that defines a locking connection. In one form, the registration takes place upon the movement of the first and second sets toward one another until seated contact is made between them. Such movement takes place along a common radial axis (that is to say, dimension rather than direction) while simultaneously having the first and second locks in an axially and radially aligned placement relationship to one another.

In one form, the assembly formed by such registration may define a certain amount of axial slack S2 and radial slack S1, which both should be minimized, but large enough for ease of insertion. Radial slack S1 should be large enough to ensure that the final diameter of the lock 33 should not exceed the diameter of the tubes 20, 30, but it has to be small enough to ensure that the longitudinal axes of both tubes are as good as common. When a tension force F1 is applied, this force will be taken by remaining wall parts 37 of sections 32 and transferred to edge 34, which will take this force entirely. Therefore, height T of edge 34 should be sufficient. However, the height of edge 34 should be chosen such, that the remaining wall parts 37 in sections 32 are also strong enough. In one form, this is optimized for each type of connection, depending on materials or combination of materials, geometry and so on.

In one form (not shown), it is possible within the sets of elongate members to provide one of them with a different outer diameter or (in the case where such elongate members are tubular) wall thickness. By way of example, the one of the elongate members that is configured as an extension member may have an outer diameter which is only slightly smaller or larger than the diameter D of the solid wire 10 of FIG. 1 or the tube 20 of FIG. 2, but with a similar lock as that depicted in wire 10 or tube 20. In this way, there will still be enough contact surface on the respective edges 14, 24 to form and maintain a locking connection that is not too weak (such as in the extension member has much smaller dimensions that differ too much from the opposing coupling section to make a proper edge contact surface).

Likewise, in a configuration where the wall thickness of an extension tube is larger than for either the first wire 10 or first tube 20. Thus, if the inner diameters are identical, the contact surface on edge 24 is as strong as for coupling tubes with identical inner and outer diameters. The given examples in the drawings of FIGS. 1 through 3 are schematically, with X=D, H1=D/3, T=D/3 and H2=2D/3 (without taking slack S1 in consideration here).

When a compression force F2 is applied, edges 36 will take up this force, after the gaps with a width equal to axial slack S2 are closed.

The connection as shown in FIG. 3 cannot take torsional moments, because the locks would disengage when edges 34 move apart. In this position lock 33 acts as a hinge that cannot take bending or torsion forces.

The edge 34 may be oriented perpendicular to the length axis to prevent radial unlocking movement upon loading when assembled, while it is easy to insert this coupling. In one form, the edges 34 may have a shape that differs from the flat surface (perpendicular to the length axis), as shown so far. In that case the two parts that have to be coupled may not have edges 34 with identical geometry. These different shapes may fit in such a way that torque or bending forces can be taken as well. Eventually there is a need for enlarging the axial slack S2 to enable assembly of such special embodiments, which may be a disadvantage, dependent on the application. The orientation of edge 34 may have an angle with the length axis that differs from 90 degrees.

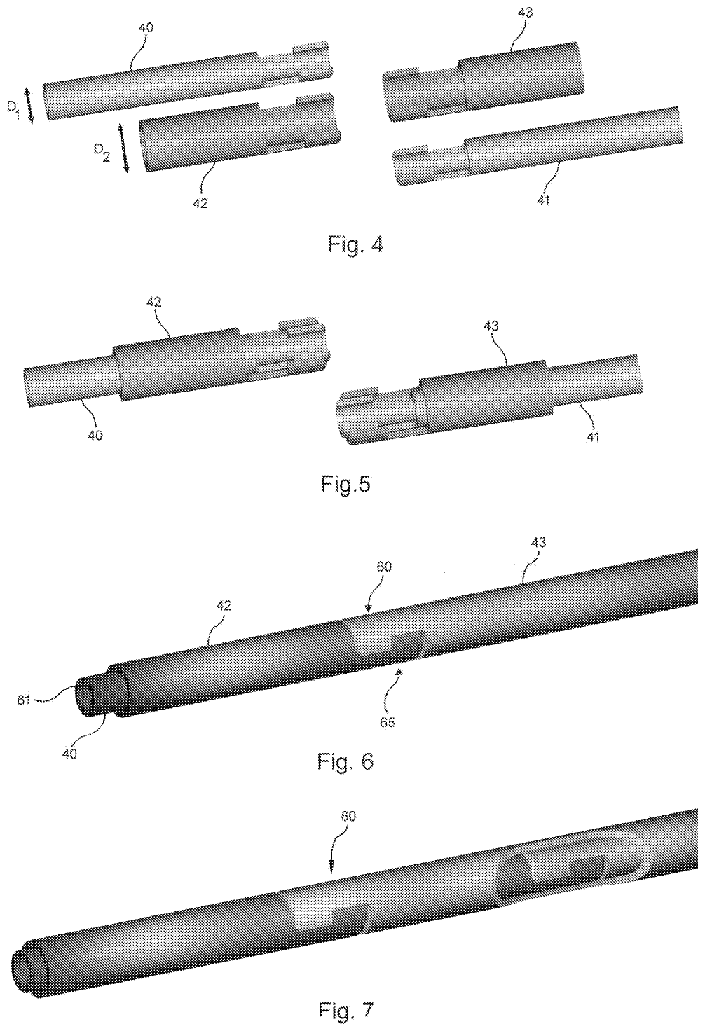

Referring next to FIG. 4, two sets of tubes are shown that may be locked together. In one form, the tubes 40, 41 and 42, 43 of each of the first and second sets are of generally similar construction to the tubes 20, 30 of FIGS. 2 and 3. The first set of tubes 40 and 41 have diameter D1, while the second set of tubes 42 and 43 have diameter D2, while all are elongate members (also referred to herein as elongate devices or elongate elements) due to their axially elongate profile. The first set is sized to fit inside the second, with enough tolerance to move radially or tangentially relative to one another with ease. In a manner similar to FIG. 2, in an alternate embodiment one or both of the tubes 40, 41 may be replaced with a comparably-sized solid wire 10. In one form, all four tubes 40, 41, 42, 43 are provided with similar coupling sections in a manner similar to the coupling sections 21, 32 of FIGS. 2 and 3. As will be discussed in more detail as follows, such coupling sections may be used to form a lock or similar engagement between joined tubes, such as previously described in conjunction with FIG. 3.

Referring next to FIG. 5, cooperating elongate members from both sets of tubes of FIG. 4 are shown in a partially assembled states just before locking. In particular, tube 40 is inserted in tube 42 and their respective coupling sections are lined up together while the same is done for tubes 41 and 43. As can be seen, the coupling section that is formed at the distal end of tube 40 is sized and shaped to form a lock when joined to the coupling section that is formed at the proximal end of tube 41 while the same applies to the coupling sections of tubes 42 and 43. This is the only assembly position in which the connection can be made or unmade. The coupling sections of the tubes 40 and 42 that are shown on the left side of the figure are aligned along the same elongate axis to enable the axially aligned coupling sections of the opposing tubes 41 and 43 that are shown on the right side of the figure to be hooked simultaneously into the locks through the aforementioned registration process. As will be appreciated, the registration to create the first and second locks takes place simultaneously by having all four of the coupling sections be hooked together in one single movement.

Referring next to FIG. 6, an assembly 60 of both sets of tubes are shown connected to one another. As can be seen, both locks are situated at the same axial location 65. The tubes 41 and 43 of FIG. 5 now have been rotated 180 degrees around their elongate (that is to say, length) axis, and simultaneously inserted radially into the lock formed by tubes 40 and 42. In this position the connection can easily be reversed or canceled by lifting the tube 43 out of the tube 42 along a radial direction. Contrarily, if the first set of tubes 40 and 41 is rotated tangentially (that is to say, circumferentially) over an angle that is different from a multiple of 360 degrees, disconnection becomes impossible as the radial direction for such disconnection of the first set of tubes 40 and 41 is different from the direction for the second set of tubes 42 and 43. This means that the first set prevents disconnection for the second set and vice versa. At this stage of connectivity, the joining is still not optimal, because location 65 acts as a hinge that by being relatively incapable of taking a bending force is at risk of disconnection, such as through accidental rotation or the like. A lumen 61 that extends axially through the tubes 40 and 41 defines a conduit-like passageway that in one form has a smooth surface over its length, even inside the lock near location 65. This in turn facilitates the insertion of various kinds of additional devices (such as those used in medical procedures as will be discussed later) into the assembly 60. In a similar manner, the outer surface defined by the assembly 60 may be made smooth to enable the sliding of additional devices thereover in ways that would be much more difficult to achieve if other connections or related protrusions such as welding, soldering, adhesives or the like were used at location 65 By way of example, for a medical device or assembly that is used for a steerable guidewire, like the one with the aforementioned DTM, the smoothness of the inner diameter of the joined inner tubes 40, 41 promotes ease of insertion of a pull wire for steering the bendable distal tip through the entire length of such device or assembly.

In one form, indicia may be placed on the outer surface of tubes 40 and 42 (as well as tubes 41 and 43, if needed). In one form, such indicia includes markers in order to display the only position in which assembly or disassembly between the tubes is possible, for example, when the markers are lined up. Securing the locks can be achieved by changing the relative position of these markers. For example, in the case of the assembly 60, such securing is achieved by relative rotation.

Referring next to FIG. 7, a schematic view on the assembly 60 of FIG. 6 is shown, wherein a portion of the wall that makes up the second set and its outer tubes 42, 43 is depicted in partial cutaway in order to show the different axial position of the locks in the set of inner tubes 40, 41. In this position (where the relative axial placement of the inner and outer sets of tubes differs from that depicted in FIG. 6, such as by sliding between them along the axial direction), the assembly 60 cannot be opened as the joined inner tubes 40, 41 prevent the release of the lock in the outer tube and vice versa. Both connections are stable enough to transmit pulling and pushing forces as well as torque over the length of each of the tubes 40, 41, 42, 43, while the possibility of axial sliding and tangential rotation between inner and outer tube sets remains possible. Moreover, the assembly 60 can be bent without unlocking the individual elongate members. This bending strength of the assembly 60 is sufficient as long as the axial distance of both locking sets is large enough.

If needed, the disclosed connection can be made temporary by using a releasable clamp (not shown) that connects one of the outer tubes 42, 43 to a corresponding inner tube 40, 41 on any (remote) axial location. After loosening the clamp, the entire assembly 60 can be disassembled by sliding the inner tubes 40, 41 back to the position as shown in FIG. 6 and rotating the lock to the proper tangential angle for opening. In one form, this can be seen from the outside by lining up the markers that were previously discussed. If present, the optional clamp that creates the axial fixation of the distance between the locking sets may be located in a remote location, such as at or near the proximal end.

After having placed the joined tubes that make up the first and second sets in the position as shown in FIG. 7, a permanent locking connection may be formed, such as through a simple spot weld, crimp or glue joint. Such affixing may be made at any suitable location along the length of the assembly 60, such as near the distal end in order to create a stable, reliable, permanent and mono-diameter connection.

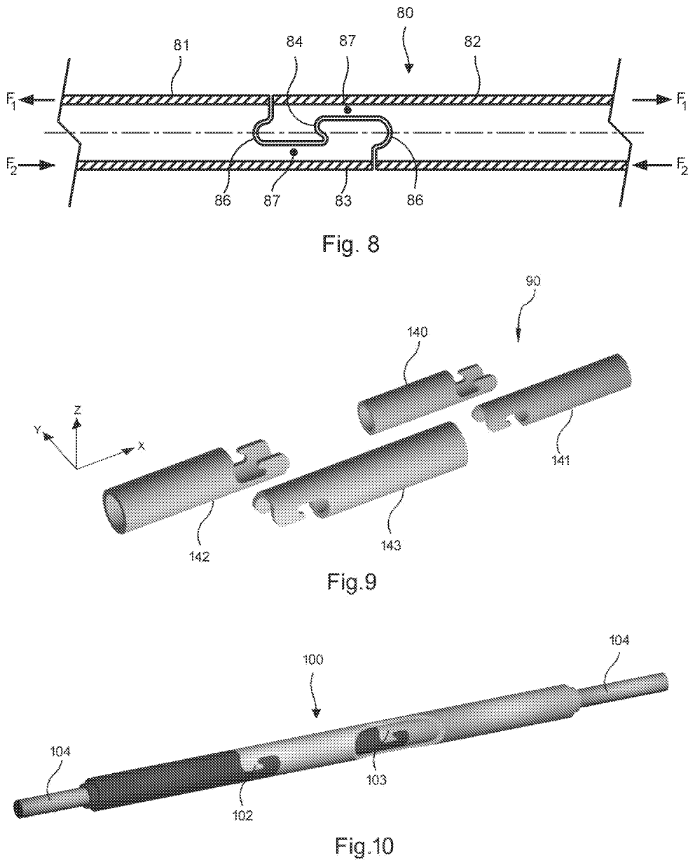

Referring next to FIG. 8, a set 80 of two tubes 81 and 82 are shown in an assembled state to form a lock 83. As can be seen, the contour of the lock 83 differs from the contour of the lock described in conjunction with FIG. 3. In particular, unlike the generally flat contact profile of the edges 34 and 36 in FIG. 3, edges 84 and 86 have a curved contour that can also take up forces in a direction perpendicular (that is to say, along the radial direction) to the length axis of the device, which prevents excessive bending of the tube ends at their weakest wall sections 87 upon applying tension force F1. When configured with the non-planar contact surface such as in FIG. 8, the registration process that takes place in FIG. 3 may require some slight fine-tuning or related adjustment. In particular, while registration of the tubes 20, 30 of FIG. 3 may take place upon pure radial movement along the precise common radial axis between them until a seated interference-based contact with one another is made, it will be appreciated that the overhang produced by the non-planar contact surfaces of the tubes 81, 82 may require a slight amount of pivoting or related adjustment prior to such seating contact being made. It will be appreciated that the degree of precision required (with or without such additional pivoting) for such radial movement in order to produce the registration and consequent locking varies according to the context, and that such minute differences are both deemed to be within the present disclosure and the claims unless noted otherwise.

When a tension force F1 is applied, the contact surfaces that are defined by the edges 84 can take up this force and a compression force F2 is taken up by the contact surfaces that are defined by the edges 86. These different contour shapes may fit in such a way that they will not easily disengage and further are capable of taking up torque or bending forces as well. Although not shown, the edges 84 and 86 may have other contour shapes as well, dependent on the needed use and ways of assembly.

Referring next to FIG. 9, two sets 90 of tubes are shown before they are locked together in a manner generally similar to that of the two sets of tubes of FIG. 4. As can be seen, the coupling sections that are used to make up the first and second locks differ from the coupling sections of the tubes of FIG. 4 in that the present have contact surfaces that define a curved, non-planar contour. In construction, the outer tube 142 and its inserted inner tube 140 that form a first set must be placed relative to the outer tube 143 and its inserted inner tube 141 that form a second set in order to form the pair of locks after which both sides are rotated to a mounting position in a manner similar to that of FIG. 5. As with the tubes of FIGS. 4 and 5, the tubes 140, 141, 142 and 143 are elongate members defined by their axially elongate profile. While the locks of FIGS. 1 through 7 can be hooked up in several directions, the curved lock edges can only be assembled or disassembled by sliding them together or apart in a Y-direction. In a Z-direction the curvature of the edges prevents disengagement. Any element that prevents the disengagement in the Y-direction can be used to secure the locking. Such an element can be an external tube or sleeve, or an internal wire or tube.

Referring next to FIG. 10, a schematic view of the two sets of tubes 90 of FIG. 9 is shown placed into an assembly 100. A lock 102 is shown engaged in the second set that is formed by the outer tubes 142, 143. As with the assembly 60 of FIG. 7, a portion of the wall that makes up the second set is depicted in partial cutaway in order to show the different axial position of the locks in the first set that is formed by the inner tubes 140, 141. In one form, the extra locking functionality that is enabled by the curved lock contours and corresponding enhanced radial interference fit between the cooperating tubes of each set may be stronger than that of the embodiment of FIGS. 1 through 7, and the choice of which may be made to depend on manufacturing costs, end-use needs or the like. A control wire 104 can be inserted to extend over at least the entire axial direction of the assembly 100, including from the proximal end to the distal end. It will be appreciated that the control wire 104 may be moved back and forth along the elongated axial direction, such as through sliding through an inner lumen that is formed in the first set that is made up of the inner tubes 140, 141.

It will be appreciated that it may not always be necessary to use a double set of locks as shown and described herein, and that a set with three or more concentric elongate elements (in the form of corresponding third sets, fourth sets and so forth) may be used in the same manner. It will be appreciated that in the third and subsequent sets, none, some or all of the locks that are formed by the first and second sets of FIGS. 1 through 10 need be present. In any event, each concentric layer of elements outside of the innermost layer defines-out of necessity-tubular cross-sections, while such innermost layer may be either tubular, solid or a hybrid of both. Alternatively, a single set of locks may be used. In this latter form, either the inner or outer elongate members may be replaced by a continuous elongate member that is devoid of the locks disclosed herein, yet may be configured to secure the connection of the elongate members disclosed herein that do possess such locks. All such variants employing one, two or more sets are deemed to be within the scope of the present disclosure. Relatedly, in one form, a connection formed by two joined tubes with a lock as described herein can be secured by inserting a wire or tube (neither of which are shown) that has no lock. In such a configuration, unlocking is only possible after removal of the inner wire or tube. Furthermore, in another form, the use of two inner tubes (such as tubes 40, 41 or tubes 140, 141) with locks as described may be used to make a connection, and then secured by putting these locked tubes inside an uncut outer tube (not shown). Thus, the extension system may have one or the other of its first or second sets of tubes with their respective inner or outer elongate members be replaced by a continuous elongate member without locks in order to form the stabilized connection of the other set of joined elongate members that does include the locks.

In one form, an extension system for coupling elongate devices includes a first set of elongate members and a second set of elongate members. The first set includes inner elongate members each with proximal and distal ends, wherein one of the inner elongate members defines a coupling section at its distal end while the other of the inner elongate members defines a coupling section at its proximal end such that upon joining the inner elongate members, their coupling sections form a first lock. The second set includes outer elongate members each with proximal and distal ends, wherein one of the outer elongate members defines a coupling section at its distal end while the other of the outer elongate members defines a coupling section at its proximal end such that upon joining the outer elongate members, their coupling sections form a second lock, the second set of tubes being sized and shaped such that each inner elongate member may be placed within a corresponding outer elongate member to be axially and tangentially moveable therein, the first and second sets of elongate members being cooperative with one another such that upon assembly of the first and second sets of elongate members into the extension system and movement of the first and second locks relative to one another, a stabilized connection of the extension system is formed. The placement of the first set into the second set correlates to a concentric fit such that the second set are out of necessity configured as tubes, while the first set may have one or both of tubes and wires.

In one form, a method for coupling elongate devices is disclosed. The method includes configuring a first set of elongate members each defining a coupling section formed at a substantial end thereof. The method further includes configuring a second set of elongate members to define a tubular structure that is sized and shaped such that each of the corresponding elongate members of the first set may be placed concentrically therein with axial and tangential freedom of movement between them. As with the first set, each of the elongate members of the second set defines a coupling section formed at a substantial end thereof. Thus, once the elongate members of the first set are placed into a corresponding one of the elongate members of the second set in such a way that their coupling sections are rotationally and axially aligned, and further once the other of the elongate members of the first set are placed into a corresponding other one of the elongate members of the second set to have their coupling sections be rotationally and axially aligned, and further once the first set is placed into registration with the second set so that their coupling sections form a first set lock and a second set lock, and (iv) lastly once movement of the first and second locks is made relative to one another within the extension system, the system experiences a stabilized connection (that is, one that is not easily unlocked).

In one form, a method of using an extension system is disclosed. The method includes forming numerous locks by placing a first set of elongate members into registration with a second set of elongate members wherein each of the elongate members of both sets define a coupling section formed at a substantial end thereof and the second set defines a tubular structure that is sized and shaped such that each of the corresponding elongate members of the first set may be placed concentrically therein with axial and tangential freedom of movement therebetween. The first and second sets become formed by having each of the elongate members of the first set first situated within a corresponding one of the elongate members of the second set such that their coupling sections are rotationally and axially aligned, after which the locks become stabilized by moving the first and second sets relative to one another. The method also includes placing the portion of the extension system that has the stabilized locks within a lumen (such as those used for medical, industrial or other procedures).

Devices according to the present disclosure can be produced as a kit with different distal parts and several lengths of extension wire or tubing and different materials, all with similar locks. This makes it possible to mount any combination during the procedure in only a few seconds. Advantageously, the production of the most expensive part, which is in most cases the distal section, can be simplified because this section is only long enough for its specific purpose, thus making handling during production much easier. The remainder of the length is attached later by choosing an extension from the kit. All parts in the kit may be sterilized and ready for customized assembly, such as in a hospital operating room.

While the present disclosure emphasizes a device for guidewire applications, it will be appreciated by those skilled in the art that but the same principle can be used for a range of different elongate elements for exoluminal or endoluminal applications, including catheters, steerable tips, endoscopes, laser systems, ablation systems, stents, filters, angioplasty balloons, drains, dilators, filters, baskets, filterbaskets, anchors, floating anchors, occlusion devices, guidewires, stylets, electrodes, leads, drill bits and angioplasty devices for opening chronic occlusions, catheter sheaths for use with catheter introducers or a drug infusion catheter, or related medical devices. Likewise, the device may further include one or more endoluminal devices that can slidably fit over a sheath. The endoluminal device can be at least any of a catheter, steerable tip, endoscope, stent, filter, angioplasty balloon, drain, dilator, filter, basket, filterbasket, anchor, floating anchor, occlusion device, guidewire, stylet, electrode, lead, drilling device, catheter sheath for use with catheter introducers or a drug infusion catheter, as well as combinations of the above. Similarly, the device itself may be a catheter, steerable tip, stent, filter, angioplasty balloon, drilling device, drain, dilator, basket, filterbasket, anchor, floating anchor, occlusion device, guidewire, stylet, electrode, lead, catheter sheath for use with catheter introducers or a drug infusion catheter, or combination of the above. Furthermore, materials making up the control element and sheath can be made from polymers, metals or similar structural constituents, or combinations thereof. In a particular form, the metal can be a shape-memory metal with shape memory, super-elasticity, or linear elasticity. Such shape memory metals may be made as a binary alloy, but also a ternary alloy or even contain more elements. These materials are especially valuable for applications requiring reconfigurable or related components.

As previously mentioned, the devices disclosed herein may be used in non-medical applications (such as various industrial procedures) as well as medical applications. For devices using the principles according to the disclosure that are used in industrial and other fields than medical, different sizes and different techniques for providing the slots that create the locks may be used. Some non-limiting industrial procedure examples include water jet cutting, etching, abrasive cutting, EDM, photo etching and others. In another embodiment, there is no cutting of slots, for example if a technique such as 3-dimensional printing is used to form the device with an integrated pattern of slots. In another form of the device, pipe joints may be used in oil wells, water wells, gas wells or the like, as well as for space applications or transportation systems. In yet another form, the devices may be configured as an endoscope for medical and non-medical use. It will be appreciated that the axial and radial dimensions of the extension system and associated devices discussed herein will depend on the end-use application, where endoluminal procedures for medical use may involve the use of such systems with smaller dimensions than those that are sized for lumen of industrial and other processes, and that all such sizes are within the scope of the present disclosure.

In general, it is advantageous if the distal end of a guidewire assembly is relatively compliant or floppy, while the majority of the length should be kink resistant, pushable, bendable and able to transmit torsional forces from the proximal to distal end in order to maneuver the assembly accurately. The tubular sheath can be chosen from any wire or hypotube material suitable for guidewire or catheter applications. One specifically suitable material is superelastic nitinol, a nickel-titanium alloy with shape-memory properties that is well-known for its flexibility, pushability, biocompatibility and kink resistance. In one configuration, the majority of the length of the tubular sheath may made of metal while the distal section may be made from a relatively soft and flexible material that easily deforms when the control wire being moved causes an axial compression in the tubular sheath. The control wire can be made of a high strength yet flexible polymer. If improved visibility (such as for magnetic resonance imaging (MRI) or related radio-opacity) is needed, additional markers of materials like gold, platinum, silver, tungsten, iridium or the like may be used at specific locations on either the control wire or the tubular sheath. Other material choices include metals and related materials for improved strength, stiffness or visibility for MRI or radio-opacity. Nitinol does not have to be in its superelastic mode, but can also be used in its linear elastic state, caused by a different thermomechanical production process.

There are several options to making steerable devices according to the present disclosure. Moreover, it is an object of the present disclosure that devices be used in medical procedures, comprising minimal invasive devices, surgical tools, steerable drilling tools, instruments, rotating instruments, placement of pacemaker leads and implants. It is also an object of the present disclosure that such devices may be used in non-medical procedures, including but not limited to exploration, completion and maintenance of oil, gas and water wells, fluid and gas transport systems, manipulators in robotics, vacuum environments, laboratory equipment and other fields. Even for the use outside of a lumen devices according to the present disclosure may be used, for example in a robot arm or in a manipulator in outer space or under water, like a manipulator arm on a submarine. One example would be an antenna for outer space applications. Other applications are the fine adjustment of parts in drones and related unmanned aerial vehicles, like for example fine adjustment of wing flaps, rudders or propeller blades.

The present description and accompanying figures make clear how a reliable and simple locking can be achieved, which can take bending, torsion, pushing and pulling forces for the separate inner and outer tubes, but still enable relative sliding and rotation between inner and outer tube.

It is noted that the terms “substantially”, “approximately” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

Within the present disclosure, terms such as “preferably”, “generally” and “typically” are not utilized to limit the scope of the claims or to imply that certain features are critical, essential, or even important to the disclosed structures or functions. Rather, these terms are merely intended to highlight alternative or additional features that may or may not be utilized in a particular embodiment of the disclosed subject matter.

Within the present disclosure, the use of the prepositional phrase “at least one of” is deemed to be an open-ended expression that has both conjunctive and disjunctive attributes. For example, a claim that states “at least one of A, B and C” (where A, B and C are definite or indefinite articles that are the referents of the prepositional phrase) means A alone, B alone, C alone, A and B together, A and C together, B and C together, or A, B and C together. By way of example within the present disclosure, if a claim recites that a device comprises at least one of a first component, a second component and a third component, and if such device has the first component alone, the second component alone, the third component alone or any combination of the first, second and third components, then component or components satisfies the claim.

Within the present disclosure, certain terms are used to establish a degree of connectivity or related structural, physical or other cooperation between various components, as well as between such components and users. Such terms, such as “associated with”, “corresponding to”, “cooperative with” or the like, are understood to form an exclusive or non-exclusive relationship between the components and the user or users to which they refer, and will be understood as one or the other, depending on the context.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1.-33. (canceled)

34. An extension system for coupling elongate devices, the extension system comprising:
a first set of elongate members each defining a coupling section formed at a substantial end thereof; and
a second set of elongate members defining a tubular structure that is sized and shaped such that each of the corresponding elongate members of the first set may be placed concentrically therein with axial freedom of movement therebetween, wherein each of the elongate members of the second set defines a coupling section formed at a substantial end thereof, the first and second sets configured such that a stabilized connection of the extension system is formed by taking the steps of:
(i) placement of one of the elongate members of the first set into a corresponding one of the elongate members of the second set such that their coupling sections are rotationally and axially aligned;
(ii) placement of the other of the elongate members of the first set into a corresponding other one of the elongate members of the second set such that their coupling sections are rotationally and axially aligned;
(iii) simultaneous placement of the coupling sections of both the first and second set into registration with each other so that their coupling sections form a first set lock and a second set lock; and
(iv) displacement of the first and second set locks relative to one another.

35. The extension system of claim 34, wherein the registration occurs through movement of the first and second sets toward one another until seated contact is made along a common radial dimension while simultaneously having the first and second lock sets in an axially and radially aligned placement relationship to one another.

36. The extension system of claim 35, wherein the displacement of the first and second lock sets relative to one another is through at least one of relative axial movement between the first and second sets and relative rotational movement between the first and second sets such that disconnection of the first and second coupling sections is avoided.

37. The extension system of claim 34, wherein the second set is sized and shaped to accept an elongate element placed thereover in order to prevent the first and second lock sets from becoming unlocked.

38. The extension system of claim 34, wherein at least one of the elongate members of the first set defines a tubular structure.

39. The extension system of claim 34, wherein both of the elongate members of the first set define a tubular structure.

40. The extension system of claim 34, wherein at least one of the elongate members of the first set defines a solid wire structure.

41. The extension system of claim 34, wherein the displacement of the first and second lock sets relative to one another to form the stabilized connection comprises a tangential rotation of the first set relative to the second set over an angle that differs from a multiple of 360 degrees.

42. The extension system of claim 34, wherein the displacement of the first and second lock sets relative to one another to form the stabilized connection comprises placement of the first set in a different axial location relative to the second set.

43. The extension system of claim 34, further comprising a tool that is configured to move the first set relative to the second set through at least one of axial and tangential repositioning therebetween.

44. The extension system of claim 34, further comprising a means for preventing at least one of axial and tangential repositioning between the first and second sets.

45. The extension system of claim 44, wherein the means for preventing at least one of axial and tangential unlocking comprises at least one of a spot weld, glue joint and crimp.

46. The extension system of claim 44, wherein the means are situated substantially near at least one of a proximal end and a distal end of the extension system.

47. The extension system of claim 44, wherein the means comprises a releasable set of clamps.

48. The extension system of claim 34, further comprising visible indicia formed on the extension system, the indicia corresponding to information about the relative axial position of the first and second lock sets.

49. The extension system of claim 34, wherein at least one of the elongate members that make up the first and second sets are made of different materials than a corresponding one of the other elongate members with which it forms the respective first or second lock sets, the materials comprising structural constituents comprising polymers, ceramics, metals, metal alloys, metal alloys with at least one of shape memory, super-elastic or linear elastic behavior, and combinations thereof.

50. The extension system of claim 34, further comprising a third set of elongate members placed concentrically around the second set, the third set defining a coupling section formed on a substantial end thereof.

51. The extension system of claim 34, wherein the coupling sections that make up at least one of the first and second lock sets have contact surfaces that define a planar contour.

52. The extension system of claim 34, wherein the coupling sections that make up at least one of the first and second lock sets have contact surfaces that define a non-planar contour.

53. The extension system of claim 34, further comprising at least an additional inner elongate element that is sized and shaped to be slidably disposed within a lumen that is formed by the first set.

54. The extension system of claim 34, further comprising at least an additional outer elongate element that is sized and shaped to be slidably disposed over an outer surface of the second set.

55. The extension system of claim 34, wherein the second set defines a substantially uniform outer diameter along its axial direction.

56. The extension system of claim 34, wherein the first set defines an inner lumen that comprises a substantially uniform inner diameter along its axial direction.

57. The extension system of claim 34, wherein the one of the elongate members of at least one of the first and second sets defines a different diameter than the other of the elongate members within the same of the at least one of the first and second sets.

58. The extension system of claim 34, further comprising a covering that is placed concentrically around at least a part of one of the first and second sets, the covering configured to be at least one of gas-tight and liquid-tight.

59. An elongate device, at least comprising components according to the extension system of claim 34, the device configured to perform at least one of an exoluminal or endoluminal procedure that comprises the placement of the portion of the extension system with the stabilized plurality of locks within a lumen.

60. The elongate device of claim 59, wherein the extension system is part of a medical device comprising at least one of a catheter, guiding catheter, visual inspection device, suction and flushing device, steerable tip, endoscope, laser system, ablation system, stent, filter, angioplasty balloon, drain, dilator, filter, basket, filterbasket, anchor, floating anchor, occlusion device, guidewire, stylet, electrode, lead, drill bit and angioplasty device for opening chronic occlusions, catheter sheath for use with catheter introducers or a drug infusion catheter and combinations thereof.

61. The elongate device of claim 59, wherein the extension system is part of a non-medical device, comprising an oil exploration device, water exploration device, gas exploration device, fluid and gas transport system, antenna, robotic manipulator, under water device, outer space device, vacuum environment device, laboratory equipment device and combinations thereof.

62. The device of claim 59, wherein the lumen is part of a medical procedure.

63. The device of claim 59, wherein the lumen is part of a non-medical procedure.

64. The device of claim 63, wherein the non-medical procedure comprises an industrial procedure.
